(19) United States
(12) Patent Application Publication
(10) Pub. No.: US 2023/0405163 A1
(43) Pub. Date: Dec. 21, 2023

(54) AIRCRAFT DISINFECTING DEVICE

(71) Applicants: Mark Saberton, Bonita Springs, FL (US); Matthew Saberton, Bonita Springs, FL (US); Stephen Glaudel, Hatfield, PA (US); John Hagen, Plainview, MN (US); Gary Allen, Aurora, CO (US); Gregory A. Schumacher, Plainview, MN (US); Burton Russell Cordell, Rochester, MN (US)

(72) Inventors: Mark Saberton, Bonita Springs, FL (US); Matthew Saberton, Bonita Springs, FL (US); Stephen Glaudel, Hatfield, PA (US); John Hagen, Plainview, MN (US); Gary Allen, Aurora, CO (US); Gregory A. Schumacher, Plainview, MN (US); Cordell Russell Burton, Rochester, MN (US)

(21) Appl. No.: 18/198,462

(22) Filed: May 17, 2023

Related U.S. Application Data
(63) Continuation-in-part of application No. 18/124,165, filed on Mar. 21, 2023.
Application No. 18/124,165 is a continuation-in-part of application No. 17/518,910, filed on Nov. 4, 2021.

(51) Int. Cl. A61L 2/10; A61L 2/24

(52) U.S. Cl. CPC A61L 2/10; A61L 2/24; A61L 2202/14; A61L 2202/25; A61L 2202/11; B64F 5/30

(57) ABSTRACT

The present invention provides a fixture or device which is particularly adapted for installation within a confined compartment of an aircraft, such as a lavatory, galley or cockpit, and which emits UVC within safe limits, i.e., within prescribed safe wavelengths, irradiance, and times (below the ‘Actinic Dose’) while the compartment is occupied, to continuously disinfect the interior air and prevent transmission of not only viruses, but pathogens and the like. The device is preferably a triangular prism which houses a metal core pcBoard, a controller pcBoard and at least one support frame which are fabricated from a heat dissipating metal, as is the hypotenuse of the prism.

### CROSS-REFERENCE TO RELATED APPLICATION

This application is a continuation-in-part application of co-pending U.S. patent application Ser. No. 18/124,165, filed Mar. 21, 2023, for “Aircraft Disinfecting Device,” which, in turn, is a continuation-in-part of co-pending U.S. completion application, Ser. No. 17/518,910 filed on Nov. 4, 2021, which, in turn, is a Completion Application of Provisional Patent Application Ser. No. 63/127,590, filed Dec. 18, 2020, for “Aircraft Disinfecting Device,” the disclosures of which are hereby incorporated by reference in total, including the drawing(s).

### BACKGROUND OF THE INVENTION

### 1. Field of the Invention

The present invention concerns disinfecting devices. More particularly, the present invention concerns devices for disinfecting pathogens, including viruses, bacteria, spores, and fungi. Even more particularly, the present invention concerns disinfecting devices for disinfecting pathogens for use in various mobile applications, including terrestrial vehicles, aircraft, helicopters, spacecraft and the like.

### 2. Prior Art

In the above identified co-pending applications, there is disclosed a disinfecting device for use in aircraft and other environments and, in particular, in the aircraft cabin wherein the device includes at least one LED or light bulb for emitting UV-C for inactivating various pathogens, including viruses, bacteria, spores, and fungi.

As disclosed in the co-pending applications, the device thereof is efficacious most particularly within the cabin of the craft. The device is enabled to emit a broad beam, a narrow beam, or both types of irradiance beams. The device includes means for modulating the irradiance emitted by the UV-C bulb when an animate object is detected to keep the radiance within acceptable levels.

However, certain closed compartments within the craft, such as the lavatory, galley and cockpit, create different issues in the ability to disinfect within their confined spaces, thereby enabling the use, of a very high-intensity narrow beam coupled with proper positioning, along with occupancy sensors and controls.

Thus, the present invention, as disclosed hereinbelow, is directed to the disinfecting of closed confined compartments.

### SUMMARY OF THE INVENTION

The device is particularly configured to be disposed in, or upon, a headliner of the confined compartment and at a junction with a side wall of the compartment. The device is equipped with UV-C emitting light sources, preferably, LEDs, which emit their irradiance in an approximately conical shape with full-width at half maximum (FWHM) intensity distribution ranging from about 10° to about 130°. The narrow beam may have an FWHM preferably ranging from about 20° to about 50°.

The device hereof emits its UV-C irradiance in a peak wavelength in the range of roughly 200 nanometers (nm) to 280 nm, with a FWHM of the wavelength distribution of about 10 nm. The device is fabricated such that its components, including its sensors are mounted to one or more plates which function as heat sinks.

The device includes means for modulation of the intensity of the irradiance when an animate object is detected in its sensing envelope.

For a more complete understanding of the present invention, reference is made to the following detailed description and accompanying drawing.

In the drawing like reference characters refer to like parts throughout the several views in which:

### BRIEF DESCRIPTION OF THE DRAWINGS

FIG. 1 is an exploded perspective view of the device hereof;

FIG. 2 is an exploded perspective view of the front of the metal core printed circuit board used herein;

FIG. 3 is an exploded perspective view of the rear of the metal core printed circuit metal core board;

FIG. 4 is an exploded perspective view of a control printed circuit board used herein;

FIG. 5 is a cross-sectional view of the device hereof;

FIG. 6 is a first partial cross-sectional view of the device hereof;

FIG. 7 is a second partial cross-sectional view of the device hereof; and

FIG. 8 illustrates a typical aircraft lavatory compartment with an irradiating envelope.

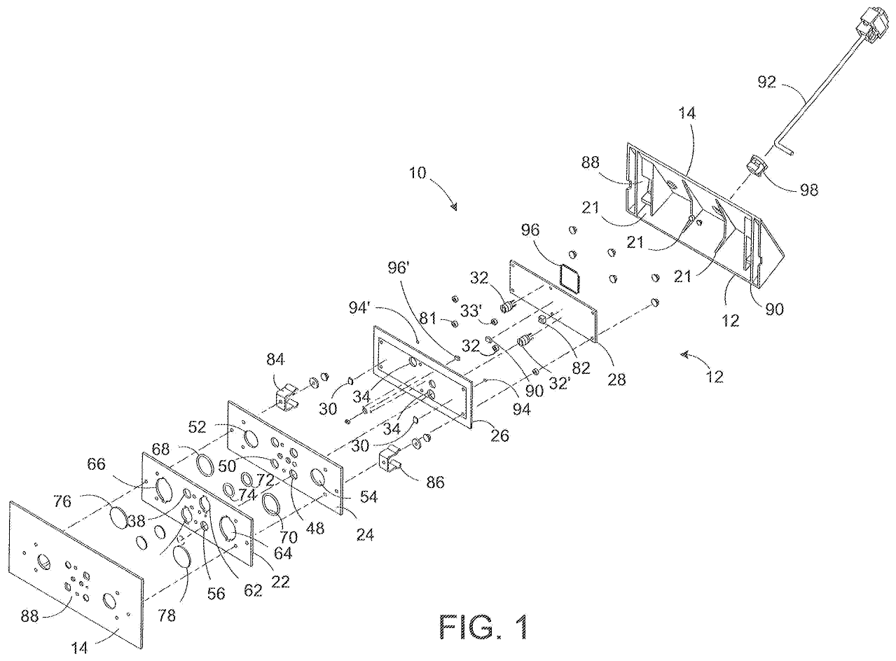

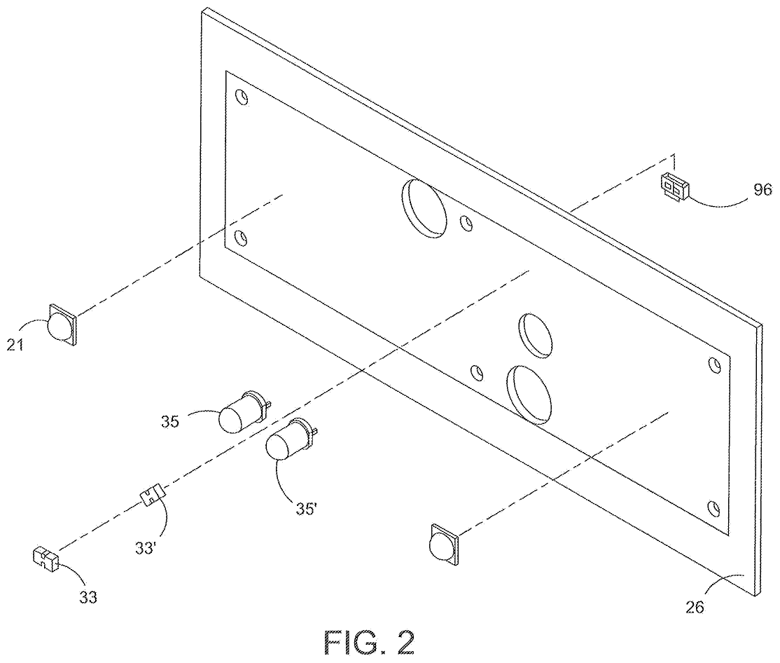

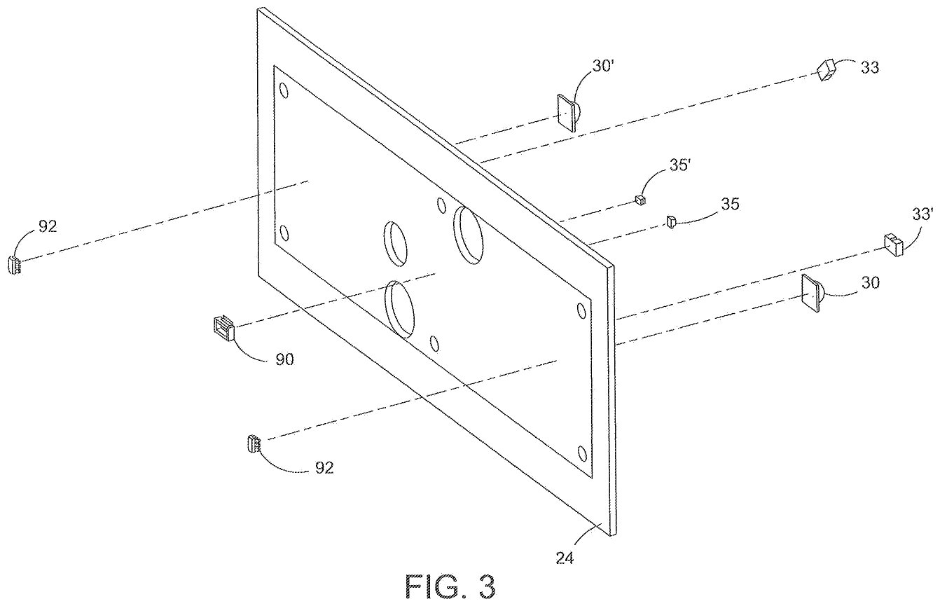

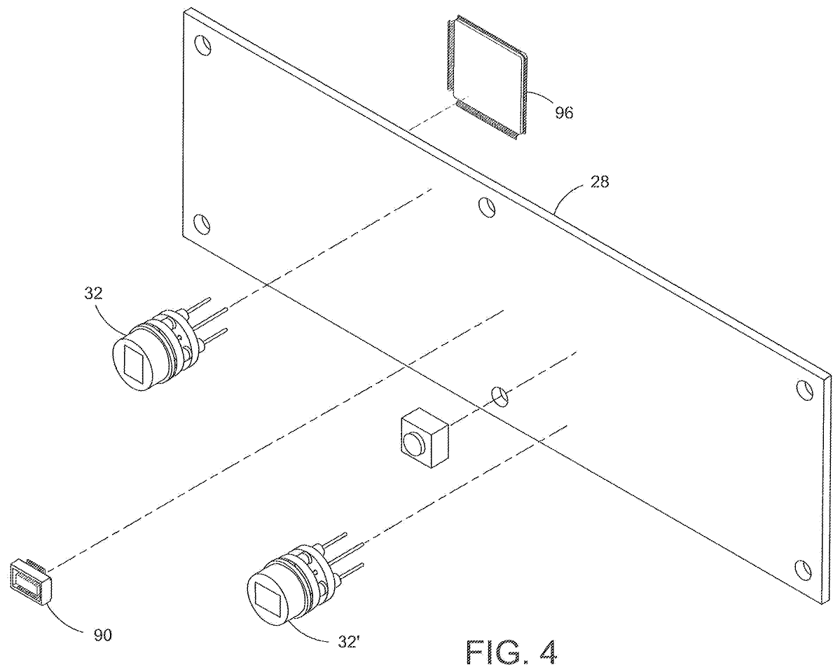

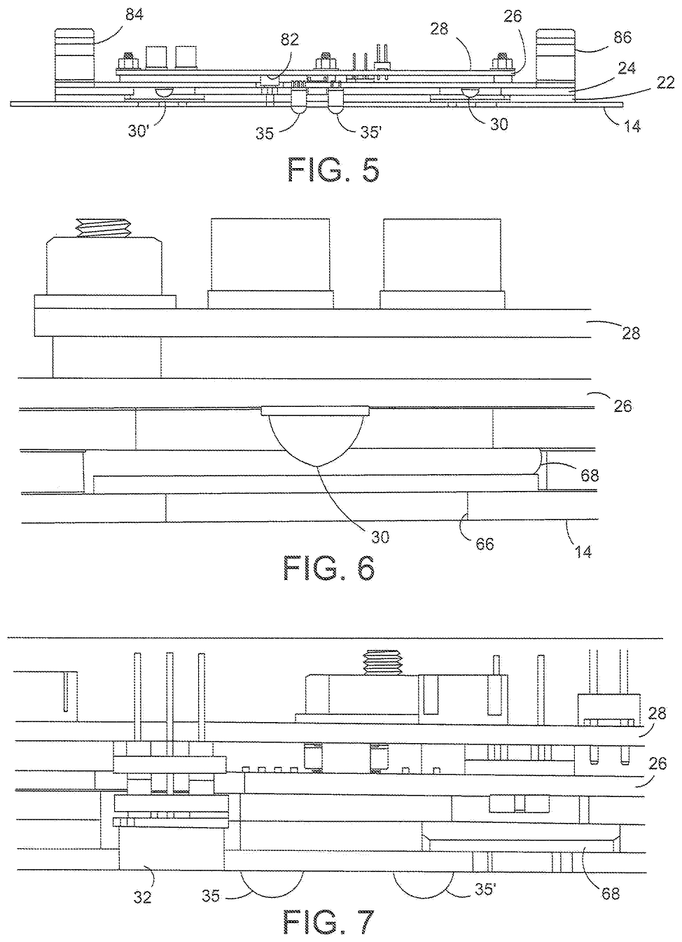

### DESCRIPTION OF THE INVENTION

Now, and with reference to the drawing, there is depicted therein an embodiment of a disinfecting device for use in a confined compartment such as an aircraft lavatory, galley or cockpit and in accordance with the present invention and, generally, depicted at 10. At the outset, it should be noted that the ensuing description is made with reference to a triangular prism housing being installed in an aircraft lavatory. However, it is to be understood that geometric configurations other than a triangular prism can be used as dictated by the design of the compartment and are within the scope hereof; e.g., having curved, or interrupted surfaces, rather than all flat surfaces.

According to this embodiment, the device, generally, comprises a substantially triangular prismatic housing 12, having a front face or wall or hypotenuse 14 and a pair of intersecting legs 16, 18. The face 14 and legs 16, 18 cooperate to form or provide an interior space 20.

The internal controls and components are disposed within the interior 20 of the housing 12.

The internal controls generally comprise at least one and, preferably, a pair of spaced apart frames 22, 24, a metal core pcBoard 26 and a separate control pcBoard 28. The metal core printed circuit board and frames are fabricated from materials which function as heat sinks. The control board is fabricated from fiberglass or other material having the requisite circuitry printed thereon and to which a microprocessor is mounted thereonto.

The present embodiment device further includes at least one UV-C emitting light source 30, which is preferably an LED. Preferably, at least a pair of spaced apart UV-C LEDs or light sources 30, 30′ are deployed herein. The LEDs 30, 30′ are mounted onto the metal core pcBoard 26.

At least one PIR sensor and, preferably, a pair of spaced apart PIR sensors 32, 32′ are mounted to the control pcBoard 26. The PIR sensors detect the presence or absence of an animate object within the sensing envelope which encompasses the UV-C beam thereof.

The present embodiment also includes at least one and preferably, a pair of pulsing sensor, such as Lidar sensors, 33, 33′. The Lidar sensors measure the distance of an animate object from the UV-C emitter and relays such information to the micro controller or microprocessor to ensure that the irradiance directed at the object is below the minimum acceptable irradiance levels, modulated, or turned off

The legs 16, 18 of the device are preferably made from any suitable resinous material such as HDPE, acrylic, or the like. Alternatively, the housing may be manufactured by any other suitable mode including additive manufacturing, injection molding, blow molding, or the like.

As shown and as noted above, according to this embodiment, the triangular prismatic configuration of the housing is defined by a 30°, 60°, 90° right triangle. The hypotenuse defines the front face 14. The direction of the LV-C emission is perpendicular to the front face, and the normal direction to the front face is 30° from the vertical direction. The 30° aim of the UV-C emission may alternately range from about 0° to about 90°, depending on the intended target of the UV-C irradiation within the closed, confined space. The sides or legs 16, 18 of the triangle and the front face or hypotenuse 14 combine together to provide an open interior 20.

Support ribs 21, 21′ are formed within the interior 20 to provide structural integrity to the legs.

The PIR sensors 32, 32′ are mounted to the control pcBoard 28. Preferably, and according to this embodiment, the PIR sensors are mounted to the pcBoard 28 and will detect animate objects at a sensing angle of about 120° from the front face of the sensor. The sensors 32, 32′ are spaced apart and are rotationally offset from each other from about 90°.

Overlying the control pcBoard 28 is the metal core pcBoard 26, which, like the front face 14 is formed from aluminum, copper, or a similar heat conducting material to function as a heat sink.

Openings 34, 34′ are provided in the metal core pcBoard 26 through which the PIR sensors 32, 32′ project.

At least one and preferably a pair of status light bulbs 35, 35′, which are optimally LEDs are also mounted to the metal core pcBoard 26.

A multi-apertured subplate 24 overlies the metal core pcBoard 26 and similarly, is formed from a suitable heat conducting material, such as aluminum or copper. The subplate 46 is provided with a plurality of apertures 48, 50, 52, 54, through which the PIR sensors, the UV-C LEDs and the status LEDs project.

Optionally and overlying the subplate 24 is a second heat sink metal subplate or frame 22, having, as shown, a plurality of apertures 56, 58, 60, 62, 64, 66 which register with the respective apertures in the subplate 46, the metal core pcBoard 26 and the control pcBoard 28.

As shown, a pair of spaced apart quartz or silicone windows 76, 78, are insertable into an associated window opening 68, 70 on the frame 22 and which align with the UV-C LEDs, as well as the status LEDs.

Interposed the frame 22 and the subplate 24 are a plurality of seals or sealing rings 68, 70, 72, 74 which correspond to the openings in the subplate and the frame. The sealing rings are compression seals which hold the respective windows in place.

A plurality of spacers which prevent any damage to the PIR sensors are interposed the metal core pcBoard and the control board.

A push button 82 is secured to the controller board and is used to calibrate the Lidar sensors. It is accessed through an opening 83.

A connector 90 is used to electronically interconnect the control pcBoard and the metal core pcBoard. The board is in communication with a suitable cable 92, and which provides power to the present device.

It should be noted that the PIR sensors are disposed in such a manner as to provide a 120° full-width sensing angle, while the UV-C bulbs provide UV-C light emission with a FWHM of about 35° around center. The emission angle of the LEDs provides a narrow beam within the confines of the closed, confined space, e.g., a lavatory. The sensing angle of the PIRs are a broad sensing angle which can extend the sensing into and around the confines of the lavatory, as well as into the cabin, itself, depending on the disposition or positioning of the device, so that the presence of an animate object just outside the door may be sensed when the door is open.

As shown in the drawing, spring clips 84, 86 secured to the interior of the front plate 14 cooperate to snap together the front plate, frame, subplate(s) and the pcBoards and snap into recesses 88,89 formed within the housing. Suitable fasteners threadably affix the components into position. Notably, there are no exposed fasteners on the front plate.

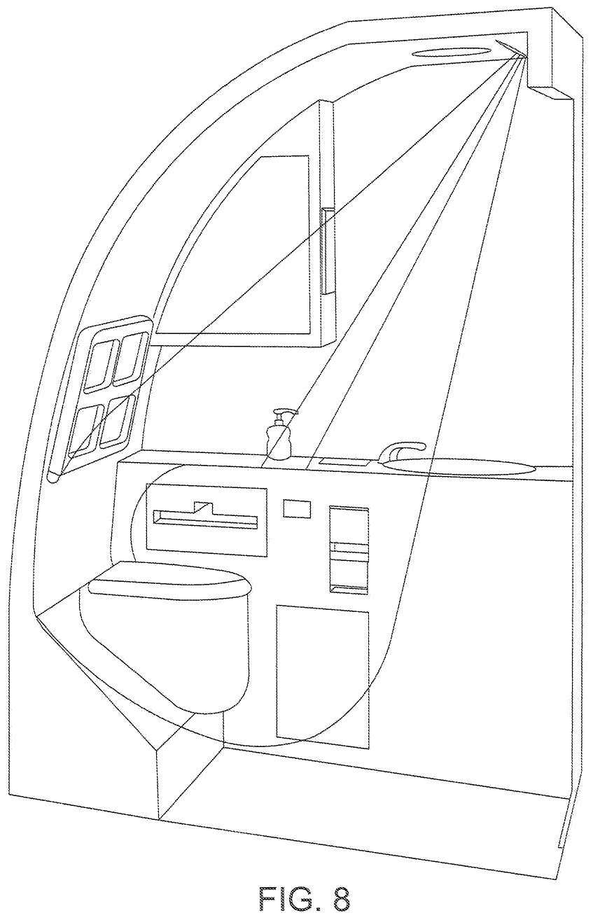

As shown in FIG. 8, in use, the device is installed proximate the ceiling of the lavatory or other confined compartment and positioned at the junction between the ceiling or headliner and the corner of a wall so that the irradiance is beamed toward the toilet, itself. It can also be directed to any area such as sink, infant changing station, or other areas of high risk pathogen exposure. Multiple UV-C LEDs, or multiple optics on or one more LEDs, may be directed toward multiple targets within the compartment.

As noted, the UV-C LEDs emit at a peak wavelength between 200 and 280 nanometers, preferably, below about 275 nanometers, more preferably below about 240 nanometers.

Optimally, the device is powered by the aircraft, itself, and which is controlled from a switch located in a secure area of the craft where only crew members or maintainers have access. The power source or cable 92 powered by the aircraft, and is plugged into the control pcBoard 28 through a plug 98 and transmits the requisite power thereto, as well as to the metal core pcBoard 26 and the controller or microprocessor 86, itself.

Although the device has been described as utilizing PIR and Lidar sensors, it is to be understood that the present device may include not only multiple similar sensors, but sensors different from the PIR and Lidar, as well.

It is to be understood that in manufacturing the present device, it must be manufactured from materials which are FAA compliant, such as for example, thermoplastics, including ABS/PVC which are durable, chemical and fire resistant, as well as explosion proof and vibration or compression resistant. Preferably, the face plate, subplates and metal core pcBoard are formed from aluminum, copper or other heat dissipating metals.

It should be noted that the present device can be modified to emit both UV-A and/or UV-C light.

In deploying the present device, when the confined compartment is occupied by passengers, the UV-C irradiance must be regulated to preclude injury to both the skin and the eyes.

In use, as a default setting, the irradiance is normally set to administer a safe ‘Actinic Dose’ (below the “Exposure Limit,” or EL) for 8-hour periods. The EL is 30 J/m², weighted by the actinic hazard function, and integrated over 8 hours. Due to the rapid repair of the protective layers of human skin and eye, the allowed exposure is renewed every 8 hours so that over a 24-hour period, e.g., for overseas flights, the EL increases to 30 J/m², per the IEC specification. The actinic weighting function provides an 8-hour EL=37 J/m² at 265 nm; EL=60 J/m² at 254 nm; and EL=100 J/m² at 240 nm.

The narrow beam of the LEDs, when used in the absence of any person or animal, will provide irradiance above the EL when the lavatory or other confined compartment is not occupied, and below the EL when occupied.

To achieve the modulation that is necessary, the sensors, when working in combination to detect the presence of a human or other animate object, will send a signal to the control or processor 96 to reduce or shut off the level of irradiance to below the maximum acceptable actinic dose level (the EL). Concurrently, the time and temperature within the environment is measured by the sensors (not numbered) and microprocessor on the control pcBoard 28 and correlated to the actinic dose to maintain the proper irradiation.

The utilization of the heat sink material, multiple temperature sensors, and LED controls built into the microprocessor enable the closed loop feedback temperature controls of the device to maintain desired temperatures below about 60° C. on the face of the housing. Thus, the front face 14, the frames 22, 24 and the metal core pcBoard 26 are, preferably, manufactured from aluminum, copper, or similar heat conducting material so that the housing and the internal mounting boards function as the heat sink to conduct heat generated by the LEDs away from the LEDs to be dissipated inot the ambient environment. By maintaining the temperature of the housing well below 60° C., the temperature of the p-n junction of the LEDs are maintained below about 800° C., thereby extending the life of the LED.

Thus, the temperature sensors are in physical contact with the metal directly connected to the LEDs and thus provide direct temperature feedback to the microprocessor on the control pcBoard to reduce the power thereto when needed to prevent premature burnout of the LEDs.

Although not shown, optionally, and in other embodiments hereof, the controller may be used for controlling the ‘Actinic Dose’ at less than the EL for an exposure time of less than 8 hours (e.g., for a short flight), so that irradiance can be set higher with this lower exposure time. By so adjusting the controller, the efficacy of the higher irradiance will disable viruses more rapidly while reducing the potential for inter-passenger infectivity. This is accomplished by calculating the total time that passengers and crew may be within the aircraft that day and increasing the irradiance by a factor of 8 divided by the number of hours. For example, if the passengers are expected to be aboard the aircraft for only 4 hours, then the irradiance may be applied at twice the level that would be allowed for an 8-hour exposure. Preferably, this is conducted by secure panel adjustments.

It is to be appreciated that if the aircraft is completely unoccupied, the UV-C LED or lamp may provide irradiance during the unoccupied time that is above the EL because there are no humans/animals in harm's way. This enables a complete ‘scrub’ of the interior of the space in a very short time, e.g., in-between flights, so as to disinfect not only any aerosols but also contaminated surfaces (i.e., ‘fomites’) such as deposited pathogens, microscopic dried mucus, blood, food, etc. Preferably, this is conducted by secure panel adjustments outside the aircraft.

According to the present invention, the tilt angle of the UV-C emitters must match the personnel sensing angle for occupants or objects approaching the galley, lavatory, cockpit or other dosed, confined compartment.

Once the presence of a human or other animate object is no longer sensed, the UV-C lamp may become operative again.

It is to be appreciated that the UV-C lamps may continue to emit its beam proximate the seated object since it is only disrupted by a person or animal within the volume of the sensing range.

As noted, the personnel detectors are preferably Lidars. However, Time-of-Flight or similar sensors, including mm wave radar sensors, or ultrasonicmm wave sensors, can be deployed with comparable efficacy as the Lidar sensors, PIR's and the like.

It should also be noted that the sensors identified herein can be deployed in various combinations depending on the space environment and design. Furthermore, in practicing the present invention, it is to be understood that for ‘fail safe’ operation, a multiplicity of sensor types (complementary) and a multiplicity of sensors (redundancy) such that any detection on any sensor will reduce irradiance to a safe level may be used herein.

Also, an excimer(s) and/or pulsed xenon discharge UV-C light source may be used in lieu of the LED emitter(s).

Further, means for emitting an audible signal may be provided and be used to emit a sound in case of “Unsafe Irradiance” along with a designated LED may be used, so that either a visually or hearing impaired passenger can know to exit the aircraft (if practical), and/or the crew can know to disconnect electrical power via a kill-switch connected to the fixture or on a control panel accessible to crew members.

The closed, confined compartment may also include a broad beam of UV-C irradiation as well as a narrow beam. An optional broad beam may have a FWHM preferably ranging from about 50 to about 130 degrees.

The present invention provides a disinfecting device for emitting UV-C irradiance within a confined space and which, generally, preferably, comprises a triangular prism, box, or rectangular shape. It is to be understood that some lavatory compartments have curved surfaces. Thus, the confined compartment may be defined in terms of a volume limit rather than shape for determining irradiance levels. For example, a volume less than about 20 m³ or less than about 10 m³ or less than about 5 m³ and/or a maximum linear dimension less than about 5 m or less than about 4 m or less than about 2.5 m, in nested claims.

It is to be further appreciated that there has been described herein a device which safely disinfects the confined spaces of an occupied aircraft against viruses including the SARS-CoV-2 virus, but also many others, such as influenza, RSV, and common cold viruses, as well as bacteria, fungi, and the like.

Having, thus, described the invention, what is claimed is:

1. An aircraft confined compartment disinfecting device comprising:
(a) a housing having a compartment facing front face and an open interior;
(b) a control pcBoard;
(c) a metal core pcBoard overlying the control pcBoard;
(d) a subplate overlying the metal core pcBoard, each of the boards and subplate being disposed within the interior;
(e) at least one UVC light mounted to the metal core pcBoard for emitting UVC irradiance through the front face into the compartment;
(f) at least one animate object detecting sensor mounted to the metal core pcBoard;
(g) at least one temperature sensor secured to the metal core pcBoard; and
(h) a microprocessor secured to the control pcBoard and in electrical communication with the animate object detecting sensor and temperature sensor, the microprosser controlling the temperature of the device and
wherein the front face, metal core pcBoard and subplate are fabricated from a heat dissipating metal.

2. The device of claim 1 wherein the housing is a triangular prism.

3. The device of claim 2 wherein the triangular prism is a right triangle, the front face defining the hypotenuse of the triangle.

4. The device of claim 3 wherein the triangle is a 30°, 60°, 90° triangle.

5. The device of claim 1 wherein the heat dissipating material is selected from the group consisting of aluminum and copper.

6. The device of claim 1 which further comprises
(a) a pulse emitting sensor for measuring distance between an animate object and the device;
(b) means for calibrating the device; and
(c) at least one status indicating light.

7. The device of claim 1 wherein the UVC emitting light is an LED, the LED emitting its beam at a wavelength ranging from about 200 to 280 nm.

8. The disinfecting device of claim 1 which further comprises at least a pair of spaced apart temperature sensors mounted to the metal core pcBoard and in electrical communication with the microprocessor controls of the LEDs, the temperature sensors and microprocessor cooperating to define a closed lop feedback temperature control to maintain the front face at a temperature below about 60° C.
